(19) United States
(12) Patent Application Publication
(10) Pub. No.: US 2023/0401747 A1
(43) Pub. Date: Dec. 14, 2023

(54) SYSTEM AND METHOD FOR CALIBRATING SPECKLE-BASED SENSOR

(71) Applicant: ROCKLEY PHOTONICS LIMITED, Altrincham (GB)

(72) Inventors: Cody DUNN, Costa Mesa, CA (US); Kate LeeAnn BECHTEL, Pleasant Hill, CA (US); James McMILLAN, Santa Monica, CA (US)

(21) Appl. No.: 18/332,639

(22) Filed: Jun. 9, 2023

Related U.S. Application Data
(60) Provisional application No. 63/351,316, filed on Jun. 10, 2022.

(51) Int. Cl. G06T 7/80; G06T 7/00

(52) U.S. Cl. CPC G06T 7/80; G06T 7/97; G06T 2207/30076; G06T 2207/20182; G06T 2207/20224; G06T 2207/20024; G06T 2207/20216

(57) ABSTRACT

A system and method for calibrating speckle-based sensor. In some embodiments, the system includes a wearable device including: a laser, an array detector; and a processing circuit, the processing circuit being configured to: obtain a calibration of the array detector, using an incoherent light source; obtain a speckle image, using the laser; and calculate a corrected speckle-based measurement, the corrected speckle-based measurement being based on the speckle image and on the calibration.

### CROSS-REFERENCE TO RELATED APPLICATION(S)

The present application claims priority to and the benefit of U.S. Provisional Application No. 63/351,316, filed Jun. 10, 2022, the entire content of which is incorporated herein by reference.

### FIELD

One or more aspects of embodiments according to the present disclosure relate to speckle-based measurements, and more particularly to a system and method for calibrating speckle-based sensor.

### BACKGROUND

Speckle-based measurements such as speckleplethysmography (SPG) may use measured speckle contrast or quantities related to the speckle contrast to infer properties of a sample. Speckle contrast measurements may be affected by factors such as shot noise, dark noise, and image vignetting.

It is with respect to this general technical environment that aspects of the present disclosure are related.

### SUMMARY

According to an embodiment of the present disclosure, there is provided a system, including: a wearable device, including: a laser, an array detector; and a processing circuit, the processing circuit being configured to: obtain a calibration of the array detector, using an incoherent light source; obtain a speckle image, using the laser; and calculate a corrected speckle-based measurement, the corrected speckle-based measurement being based on the speckle image and on the calibration.

In some embodiments, the calculating of the corrected speckle-based measurement includes correcting the speckle image for an estimated contribution from shot noise and dark noise.

In some embodiments, the obtaining of the calibration of the array detector includes: causing light from the incoherent light source to illuminate the array detector; obtaining a first image with the array detector; and obtaining a second image with the array detector, wherein the intensity in the first image is different from the intensity in the second image.

In some embodiments: the obtaining of the calibration of the array detector includes fitting a straight line to a plurality of data points including a first data point and a second data point; the first data point including: the intensity of the first image; and a standard deviation of a third image, the third image being based on the first image; and the second data point including: the intensity of the second image; and a standard deviation of a fourth image, the fourth image being based on the second image.

In some embodiments, the processing circuit is configured to form the third image by processing the first image with a high-pass filter.

In some embodiments, the high-pass filter is implemented by: processing the first image with a low-pass filter to form a low-pass filtered image, and subtracting the low-pass filtered image from the first image.

In some embodiments, the low-pass filter is a moving average filter.

In some embodiments, the wearable device includes the incoherent light source.

In some embodiments, the incoherent light source is a light-emitting diode.

In some embodiments: the wearable device includes a photoplethysmography sensor including a light-emitting diode, and the incoherent light source is the light-emitting diode of the photoplethysmography sensor.

In some embodiments, the laser includes an optical amplifier and the incoherent light source includes the optical amplifier.

In some embodiments, the incoherent light source includes the laser.

In some embodiments, the incoherent light source includes a modulator configured to modulate light generated by the laser, to reduce the coherence length of the light.

In some embodiments, the incoherent light source includes a laser drive circuit configured, in a first state, to drive the laser with a current causing single-mode operation, and, in a second state, to drive the laser with a current causing multi-mode operation.

In some embodiments: the processing circuit is further configured to measure a static scattering variance, and the corrected speckle-based measurement is further based on the static scattering variance.

In some embodiments, the measuring of the static scattering variance includes: obtaining a plurality of speckle images, using the laser; averaging the speckle images of the plurality of speckle images; and calculating a variance of the average.

In some embodiments, the system further includes a motion sensor connected to the processing circuit.

In some embodiments, the processing circuit is further configured to: detect motion exceeding a threshold, during a calibration measurement, and, in response to the detecting of the motion exceeding the threshold, repeat the calibration measurement.

In some embodiments, the processing circuit is further configured to: detect motion exceeding a threshold, during a speckle measurement, and, in response to the detecting of the motion exceeding the threshold, initiate a calibration process.

In some embodiments, the array detector is a zero-degree chief ray angle detector.

### BRIEF DESCRIPTION OF THE DRAWINGS

These and other features and advantages of the present disclosure will be appreciated and understood with reference to the specification, claims, and appended drawings wherein:

FIG. 1A is a block diagram of a wearable device and a sample, according to an embodiment of the present disclosure;

FIG. 1B is a schematic drawing of a wearable device, according to an embodiment of the present disclosure;

FIG. 1C is a schematic drawing of a wearable device, according to an embodiment of the present disclosure;

FIG. 2A is an illustration of calibration states and results, according to an embodiment of the present disclosure;

FIG. 2B is a graph of noise as a function of intensity, according to an embodiment of the present disclosure;

FIG. 3 is a graph of SPG measurements as a function of flow speed, according to an embodiment of the present disclosure; and

FIG. 4 is a flow chart, according to an embodiment of the present disclosure.

### DETAILED DESCRIPTION

The detailed description set forth below in connection with the appended drawings is intended as a description of exemplary embodiments of a system and method for calibrating a speckle-based sensor provided in accordance with the present disclosure and is not intended to represent the only forms in which the present disclosure may be constructed or utilized. The description sets forth the features of the present disclosure in connection with the illustrated embodiments. It is to be understood, however, that the same or equivalent functions and structures may be accomplished by different embodiments that are also intended to be encompassed within the scope of the disclosure. As denoted elsewhere herein, like element numbers are intended to indicate like elements or features.

A speckleplethysmography sensor may illuminate a sample with light from a coherent light source (e.g., a laser), and detect the light scattered from or transmitted through the sample with an array detector or “imaging detector” such as a complementary metal oxide semiconductor (CMOS) image sensor (CIS). The light scattered from or transmitted through the sample may form a speckle pattern on the image sensor. This speckle pattern may change if there is motion within the sample, e.g., if the sample includes blood vessels of a patient or subject and blood flows within the blood vessels. The rate at which the speckle pattern changes may be related to the motion in the sample; for example the greater the velocity of blood flow in a sample through which blood flows, the greater the rate of change of the speckle pattern may be.

Speckle contrast may be measured based on images taken with an array detector sensing the speckle pattern. For example, the measured speckle contrast may be proportional to the variance, in the pixel values, calculated across an image. If the exposure time of the array detector is sufficiently great such that the speckle pattern changes significantly during an exposure, then the measured speckle contrast may decrease because of the motion in the sample. As such, the measured speckle contrast may be used to infer characteristics of motion in the sample, such as the flow rate of blood in the sample.

Raw speckle contrast measurements, however, may not be linearly correlated to changes in blood flow over large ranges of flow speeds between 0 mm/s and 10 mm/s, which may impact the ability to compare measurements between subjects and may hinder longitudinal studies. As such, in some embodiments, sensor calibration and a correction method, based on the calibration, may be used to compute a corrected speckle-based measurement which may exhibit a more linear variation with flow velocity, and in which the influences of certain sources of error, such as shot noise and dark noise, may also be reduced. A more linear relationship between the measured, corrected speckle contrast and blood flow velocity may improve the accuracy of a variety of physiological measurements, including blood pressure.

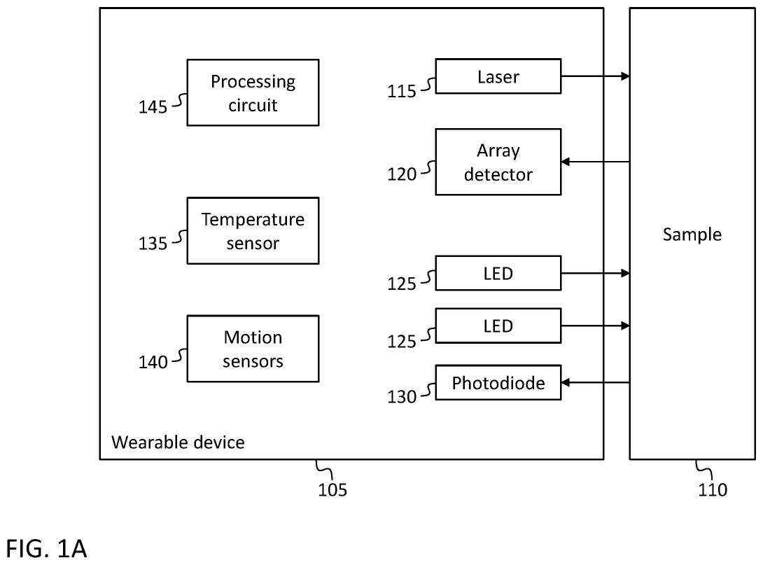

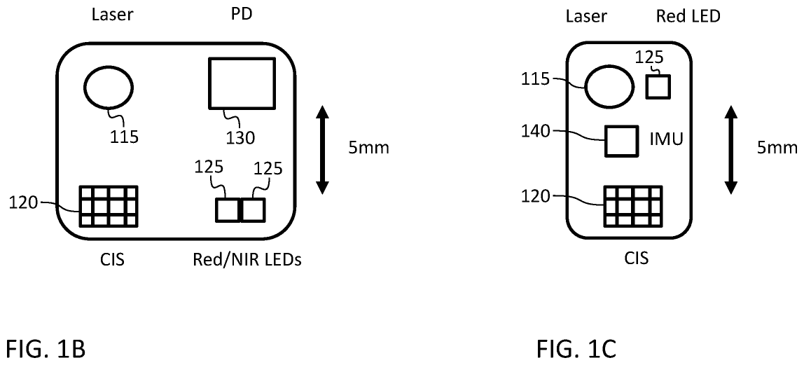

FIG. 1A shows a wearable device 105 and a sample 110, in some embodiments. The wearable device may be an instrumentation-containing enclosure that may be worn, like a wristwatch, on the wrist of a subject, or it may be similar (e.g., larger) device that may be worn on the chest or leg of the subject, or elsewhere on the subject. The wearable device may be wireless (e.g., battery powered, and using only wireless signal connections, if any). In such an embodiment, the wearable device 105 may be adjacent to (e.g., abutting against) the skin of the subject and the sample may be a portion of the skin of the subject and a portion of the tissue beneath the skin (which may include blood vessels, the flow rate within which is to be measured).

The wearable device 105 may include (i) a laser 115 and an array detector 120 for making speckleplethysmography measurements, and (ii) one or more light emitting diodes (LEDs) 125 and a photodetector (e.g., a photodiode) 130. The light emitting diodes 125 and the photodiode 130 may be used to perform photoplethysmography (PPG) measurements. The light emitting diodes 125 and the photodiode 130 may also (or instead) be used to calibrate the speckleplethysmography sensor. The wearable device 105 may also include other sensors such as a temperature sensor 135, and one or more motion sensors (e.g., accelerometers or gyroscopes) 140. A processing circuit 145 may receive sensing signals from, or control, the other elements of the wearable device 105.

FIG. 1B shows candidate layouts of components in a plan view (a top view in an orientation in which the skin of the subject, and the surface of the module that is adjacent to the skin, are horizontal). Windows in the enclosure of the wearable device 105 may allow light from the light-emitting elements 115, 125 to reach the sample 110, and may also allow light returning from the sample 110 to return to the light-detecting elements 120, 130. The light-emitting elements 115, 125 may be positioned sufficiently close to the light-detecting elements 120, 130 to allow light from any of the light-emitting elements 115, 125 to reach both of the light-detecting elements 120, 130 via a path through the sample.

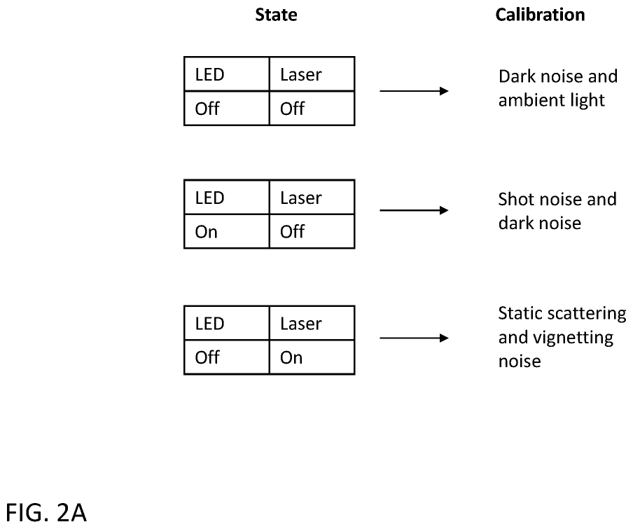

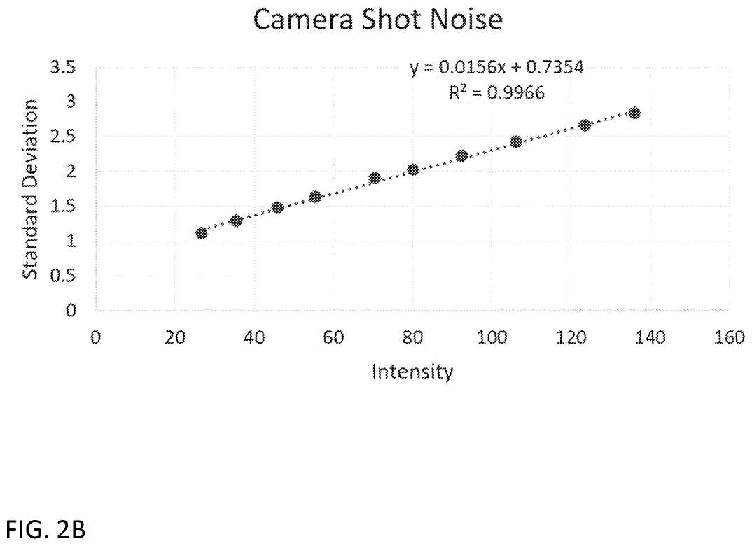

FIG. 2A shows combinations of the state of the laser 115 and the state of a light emitting diode 125 that may be used for various calibration operations. A calibration of the wearable device 105 may be performed at startup (or when the wearable device 105 is first affixed to the subject) and periodically thereafter, e.g., when triggered by any of several calibration-triggering events, as discussed in further detail below. A calibration may include one or more of (i) a dark noise and dark current measurement for the array detector 120, (ii) a shot noise measurement, (iii) an ambient light measurement, (iv) a vignetting measurement, and (v) a static scattering measurement. Ambient light intensity may be estimated by capturing an image without any of the light sources 115, 125 turned on.

Turning on an LED 125 (which may be an incoherent or non-speckle-producing source that may cause incoherent light to illuminate the array detector 120, via optical paths through the sample 110) at different intensity levels (e.g., at different drive currents) enables the calculation of shot noise relative to intensity. If the wearable device 105 includes a PPG sensor, then the shot noise calibration of the array detector 120 may be performed concurrently with an automatic gain control (AGC) sequence for the PPG sensor. For example, a light emitting diode 125 of the photoplethysmography sensor may be operated at several different levels of drive current, providing different levels of illumination to both the array detector 120 and the photodiode 130. The signal measured, during this process, by the photodiode 130, may be used to calibrate the automatic gain control of the photoplethysmography sensor.

The images obtained, during this process, by the array detector 120, may be used to perform a shot noise calibration, a dark noise calibration, or a vignetting calibration. For example, FIG. 2B shows a graph of the standard deviation (across the image) as a function of intensity. This standard deviation may be calculated by first applying a spatial high-pass filter to each image, and calculating the standard deviation of the filtered image. The high-pass filter may be a suitable infinite or finite impulse response filter; e.g., it may be implemented by subtracting from each image a low-pass filtered version of the image, the low-pass filter being a moving average filter (e.g., a five-pixel by five-pixel moving average filter).

Data such as that of FIG. 2B may be obtained by changing the drive current of the light emitting diode 125 or changing the exposure time to obtain the different intensity values (where intensity, in this context, is a measure of the total number of photons absorbed during the exposure time). Increasing the current of the light emitting diode 125 or the exposure time of the array detector 120 increases the intensity reading of the array detector 120. The average standard deviation of the pixels increases linearly with the camera intensity, as shown in FIG. 2B. A straight line may then be fit to the data, and this straight line may be used to calculate the contribution, of shot noise and dark noise, to the variance in a subsequently obtained image, based on the average intensity in the image. In some embodiments the gain of the amplifier or of the analog to digital converter of the array detector 120 may be adjusted to various different values, and a different respective calibration may be performed for each value, so that if, in operation, the gain of the array detector 120 is changed to a new gain setting (e.g., if the sample scatters an unusually high or unusually low fraction of the laser light back to the array detector 120), then the shot noise and dark noise calibration obtained for the new gain setting may be used.

The static scattering variance may be a measurement of the variance due to (i) speckle produced by static scattering and (ii) vignetting. To measure the static scattering variance, the sample 110 may be illuminated by the laser 115, forming a speckle pattern on the array detector 120. This speckle pattern may have (i) a static component due to scattering from stationary elements of the sample 110, and (ii) a changing component due to scattering from moving elements (e.g., flowing blood) in the sample 110. A plurality of images may be acquired by the array detector 120 and these may be averaged together, and a variance of the average may be calculated; this variance may be a measurement of the static scattering variance. In performing this measurement it may not be necessary to preserve the individual images; instead each may be discarded after having been added to a cumulative image (which may later be divided by the number of images that contributed to it, to obtain the average image). In the average image, the speckles due to dynamic scatterers may be averaged out, but the speckles dues to static scatterers may become clearer after averaging, if the time interval over which the images are acquired is less than the decorrelation time of the speckles due to static scattering. The static scattering image also allows the calculation of (and correction for) the variance contribution due to vignetting, which scales with intensity. Vignetting and other similar low spatial frequency sources of noise may occur due to miniaturization constraints, tissue heterogeneity, sensor fixed pattern noise, or dirt or grime covering one or more of the apertures used to control speckle size. In some embodiments, vignetting (e.g., the variance due to vignetting, or a variance (such as the static scattering variance) which includes a contribution from vignetting) is measured, and a corresponding correction is subsequently made, as discussed in further detail below. In some embodiments vignetting is instead (or also) reduced by using, as the array detector 120, a sensor (e.g., a CIS) lacking a micro-lens array. Such a sensor may also be referred to as a zero-degree chief ray angle (CRA) detector (or, e.g., as a zero-degree CRA CIS).

As mentioned above, the wearable device 105 may include a temperature sensor 135 (e.g., a thermistor), and motion sensors 140 such as an inertial measurement unit (IMU), one or more magnetometers, one or more gyroscopes, or one or more accelerometers. The temperature calculated from the thermistor may be used to improve calibration or to perform estimation of Brownian motion (which may affect the measured speckle contrast). The accelerometer may be used to determine the quality of the calibration; e.g., if there is too much movement, it may be determined that motion artifacts are likely to have degraded the quality of the calibration, and the calibration steps may be repeated. If the wearable device 105 includes a photodiode 130, then the photodiode 130 may also be used during calibration to confirm the intensity observed (e.g., for different light-emitting elements 115, 125 or for different intensities produced by the light-emitting elements 115, 125) by the array detector 120, or to help set the initial exposure time of the array detector 120. The additional sensors may also be used to determine when it is time for SPG measurement re-calibration, such as use of the IMU to determine that the subject is in a new body position.

In some embodiments, a new calibration may be performed when the wearable device 105 is first turned on (as mentioned above), when the quality of the images obtained by the array detector 120 is not acceptable, when excessive motion of the wearable device 105 relative to the sample is detected (e.g., by a change in a speckleplethysmography measurement, a change in a photoplethysmography measurement, or a signal from a motion sensor 140), when a body position change of the subject is detected (e.g., by a change in a speckleplethysmography measurement, a change in a photoplethysmography measurement, or a signal from a motion sensor 140), when a change in perfusion is detected (e.g., by a change in a speckleplethysmography measurement), when a change in blood volume is detected (e.g., by a change in a photoplethysmography measurement), when a set interval of time has elapsed since the most recent calibration, or when a user (e.g., the subject) initiates a calibration. In some embodiments some of the trigger conditions listed above may not result in a full recalibration of the wearable device 105; for example the shot noise and dark noise calibration may not be repeated each time a calibration is performed. The calibrations may be performed discretely (e.g., when triggered by one of the conditions listed above) to reduce power consumption, or continuously to improve accuracy. Image quality assessments may include quantifying saturated pixels (potentially due to changes in ambient light), quantifying pixels below the dark current, or assessing image heterogeneity outside of the expected vignetting (potentially due to dirt obscuring part of the CIS).

When the calibrations described above have been performed, they may be used to calculate a corrected speckle-based measurement. As used herein, a “speckle-based measurement” is any measurement based on speckle in one or more images obtained by an array detector. For example, a corrected speckle-based measurement may be a corrected speckle contrast K_{Linearized}, which may be calculated using the following equation:

$${K}_{Linearized} = \frac{\sqrt{{σ}_{Image}^{2} - {σ}_{Static}^{2} - {σ}_{Shot}^{2}}}{〈 {I}_{Image} 〉 - 〈 {I}_{Dark} 〉} \qquad ( 1 )$$

where σ_{Image}² is the variance in the image, σ_{Static}² is the static scattering variance, σₛₕₒₜ² is the variance due to shot noise and dark noise, <I_{Image}> is the average intensity in the image, and <I_{Dark}> is the average dark-current equivalent intensity in the image. In some embodiments, additional corrections may be made in an analogous manner; for example, the variance due to read noise, and the variance due to any nonuniformity in the ambient light may also be subtracted from variance in the image. As another example, the average intensity due to ambient light may also be subtracted from the average intensity in the image.

The corrected speckle contrast may be used to calculate a linearized SPG measurement (which is another example of a corrected speckle-based measurement), defined as follows:

$$S P {G}_{Linearized} = \frac{1}{2 T {K}_{Linearized}^{2}}$$

where T is the exposure time.

In some embodiments, the measurements and corrections described above are performed on sub-images, or “blocks” of the images obtained by the array detector 120. For example, a corrected speckle contrast may be calculated for each block, and the results for all the blocks may be averaged together. In another embodiment, a respective value of each of the quantities on the right-hand side of Equation 1 above is calculated for each block, an average value of each of these quantities is calculated (by averaging over all of the blocks) and the average values are used in Equation 1 to calculate the corrected speckle contrast. The linearized SPG measurement may then be calculated from the average corrected speckle contrast. The blocks may have sizes in pixels, of 5×5, 7×7, 16×16, 30×40, 16×16, or 480×640, for example, or the entire set of pixels of the array detector 120 may be treated as a single block. In some embodiments, the blocks may be overlapping. In some embodiments, the temporal speckle contrast is calculated for one pixel at a time; in such an embodiment the temporal speckle contrast may be corrected based on some of the calibrations described herein (e.g., based on the shot noise and dark noise calibration).

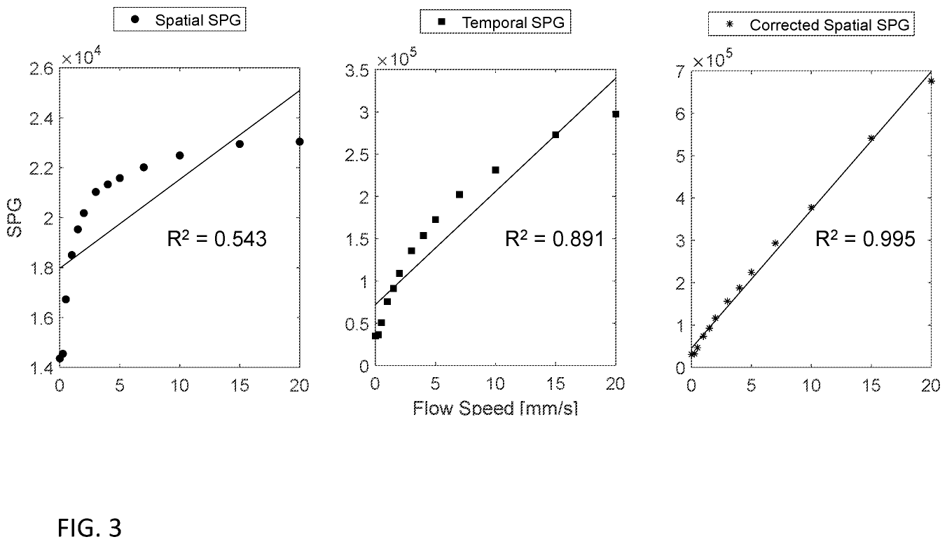

FIG. 3 shows, as a function of flow velocity, the linearized SPG measurement (in a curve labeled “Corrected Spatial SPG”) and analogous SPG measurements calculated from (i) the uncorrected speckle contrast (in a curve labeled “Spatial SPG”) and (ii) the temporal speckle contrast (in a curve labeled “Temporal SPG”). It may be seen (e.g., from the respective R² values with which the curves are labeled) that the fit of a straight line to the data is significantly better for the linearized SPG measurement than for the other two data sets.

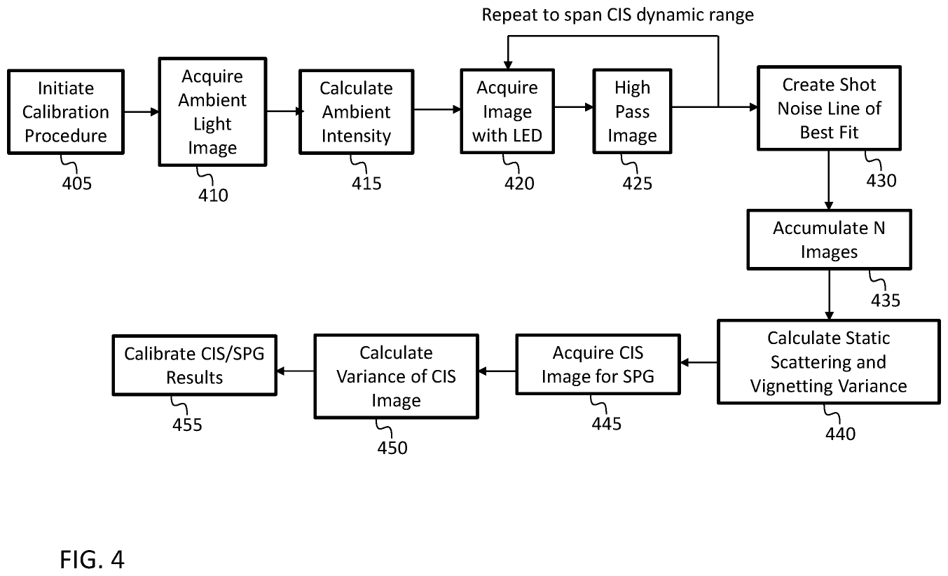

FIG. 4 shows a flow chart of a method, in some embodiments. The method includes initiating the calibration procedure, at 405, acquiring an ambient light image at 410, and calculating the ambient light intensity at 415. The method further includes repeatedly acquiring images at 420 and processing each image with a high-pass filter at 425, at different intensities (e.g., different drive currents applied to a light emitting diode 125, or different exposure times) until enough images have been obtained to span the dynamic range of the array detector 120 or to produce a reliable straight-line fit to the data obtained. The method further includes creating, at 430, a shot noise line of best fit, accumulating, at 435, a plurality of (e.g., N) images, and calculating, at 440, from the plurality of images, the variance due to static scattering and vignetting (which may be referred to as the static scattering variance). The method further includes acquiring, at 445, an image for a speckleplethysmography measurement, calculating, at 450, the variance of the image, and calibrating the results (e.g., calculating a corrected speckle-based measurement) at 455.

Any incoherent light source and coherent light source pairing may be used to replace the LED 125 and laser 115, respectively. Another embodiment may utilize a single laser (e.g., a vertical cavity surface emitting laser (VCSEL)) as the coherent source and that same laser modulated (e.g., phase modulated) as the incoherent source. Another embodiment may include a photonic integrated circuit (PIC)-based device that allows for direct control of the laser cavity (e.g., a tunable grating which acts as the output mirror of the laser and which may be detuned so that the device operates below threshold when incoherent light is needed) which offers either a lasing output or the broadband reflective semiconductor optical amplifier (RSOA) amplified spontaneous emission (ASE) output. In another embodiment a laser is used that can be driven (i) by a sufficiently high current to cause multi-mode (short coherence length) operation, or (ii) by a sufficiently low current to cause single-mode (long coherence length) operation. In some embodiments, external cavity means for on-demand reduction of speckle, such as a deformable mirror or optical phase array, are used as needed to reduce the coherence of the light.

In some embodiments, the in vivo changing blood volume does not impact the measurements because the exposure time is much shorter than the pulse period. As such, it may not be necessary for acquisitions to be timed with the pulse, and in vivo human tissue may be used for in situ device calibration. This may be preferable to performing calibration on an external fixture or stable tissue phantom.

As used herein, “a portion of” something means “at least some of” the thing, and as such may mean less than all of, or all of, the thing. As such, “a portion of” a thing includes the entire thing as a special case, i.e., the entire thing is an example of a portion of the thing. As used herein, when a second quantity is “within Y” of a first quantity X, it means that the second quantity is at least X−Y and the second quantity is at most X+Y. As used herein, when a second number is “within Y %” of a first number, it means that the second number is at least (1−Y/100) times the first number and the second number is at most (1+Y/100) times the first number. As used herein, the word “or” is inclusive, so that, for example, “A or B” means any one of (i) A, (ii) B, and (iii) A and B.

Each of the terms “processing circuit” and “means for processing” is used herein to mean any combination of hardware, firmware, and software, employed to process data or digital signals. Processing circuit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs). In a processing circuit, as used herein, each function is performed either by hardware configured, i.e., hard-wired, to perform that function, or by more general-purpose hardware, such as a CPU, configured to execute instructions stored in a non-transitory storage medium. A processing circuit may be fabricated on a single printed circuit board (PCB) or distributed over several interconnected PCBs. A processing circuit may contain other processing circuits; for example, a processing circuit may include two processing circuits, an FPGA and a CPU, interconnected on a PCB.

As used herein, when a method (e.g., an adjustment) or a first quantity (e.g., a first variable) is referred to as being “based on” a second quantity (e.g., a second variable) it means that the second quantity is an input to the method or influences the first quantity, e.g., the second quantity may be an input (e.g., the only input, or one of several inputs) to a function that calculates the first quantity, or the first quantity may be equal to the second quantity, or the first quantity may be the same as (e.g., stored at the same location or locations in memory as) the second quantity.

The terminology used herein is for the purpose of describing particular embodiments only and is not intended to be limiting of the inventive concept. As used herein, the terms “substantially,” “about,” and similar terms are used as terms of approximation and not as terms of degree, and are intended to account for the inherent deviations in measured or calculated values that would be recognized by those of ordinary skill in the art.

Any numerical range recited herein is intended to include all sub-ranges of the same numerical precision subsumed within the recited range. For example, a range of “1.0 to 10.0” or “between 1.0 and 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Similarly, a range described as “within 35% of 10” is intended to include all subranges between (and including) the recited minimum value of 6.5 (i.e., (1−35/100) times 10) and the recited maximum value of 13.5 (i.e., (1+35/100) times 10), that is, having a minimum value equal to or greater than 6.5 and a maximum value equal to or less than 13.5, such as, for example, 7.4 to 10.6. Any maximum numerical limitation recited herein is intended to include all lower numerical limitations subsumed therein and any minimum numerical limitation recited in this specification is intended to include all higher numerical limitations subsumed therein.

Although exemplary embodiments of a system and method for calibrating a speckle-based sensor have been specifically described and illustrated herein, many modifications and variations will be apparent to those skilled in the art. Accordingly, it is to be understood that a system and method for calibrating a speckle-based sensor constructed according to principles of this disclosure may be embodied other than as specifically described herein. The invention is also defined in the following claims, and equivalents thereof.

What is claimed is:

1. A system, comprising:
a wearable device, comprising:
a laser,
an array detector; and
a processing circuit,
the processing circuit being configured to:
obtain a calibration of the array detector, using an incoherent light source;
obtain a speckle image, using the laser; and
calculate a corrected speckle-based measurement, the corrected speckle-based measurement being based on the speckle image and on the calibration.

2. The system of claim 1, wherein the calculating of the corrected speckle-based measurement comprises correcting the speckle image for an estimated contribution from shot noise and dark noise.

3. The system of claim 2, wherein the obtaining of the calibration of the array detector comprises:
causing light from the incoherent light source to illuminate the array detector;
obtaining a first image with the array detector; and
obtaining a second image with the array detector,
wherein the intensity in the first image is different from the intensity in the second image.

4. The system of claim 3, wherein:
the obtaining of the calibration of the array detector comprises fitting a straight line to a plurality of data points including a first data point and a second data point;
the first data point including:
the intensity of the first image; and
a standard deviation of a third image, the third image being based on the first image; and
the second data point including:
the intensity of the second image; and
a standard deviation of a fourth image, the fourth image being based on the second image.

5. The system of claim 4, wherein the processing circuit is configured to form the third image by processing the first image with a high-pass filter.

6. The system of claim 5, wherein the high-pass filter is implemented by:
processing the first image with a low-pass filter to form a low-pass filtered image, and
subtracting the low-pass filtered image from the first image.

7. The system of claim 6, wherein the low-pass filter is a moving average filter.

8. The system of claim 1, wherein the wearable device comprises the incoherent light source.

9. The system of claim 8, wherein the incoherent light source is a light-emitting diode.

10. The system of claim 8, wherein:
the wearable device comprises a photoplethysmography sensor comprising a light-emitting diode, and
the incoherent light source is the light-emitting diode of the photoplethysmography sensor.

11. The system of claim 8, wherein the laser comprises an optical amplifier and the incoherent light source comprises the optical amplifier.

12. The system of claim 11, wherein the incoherent light source comprises the laser.

13. The system of claim 12, wherein the incoherent light source comprises a modulator configured to modulate light generated by the laser, to reduce the coherence length of the light.

14. The system of claim 12, wherein the incoherent light source comprises a laser drive circuit configured, in a first state, to drive the laser with a current causing single-mode operation, and, in a second state, to drive the laser with a current causing multi-mode operation.

15. The system of claim 1, wherein:
the processing circuit is further configured to measure a static scattering variance, and
the corrected speckle-based measurement is further based on the static scattering variance.

16. The system of claim 15, wherein the measuring of the static scattering variance comprises:
obtaining a plurality of speckle images, using the laser;
averaging the speckle images of the plurality of speckle images; and
calculating a variance of the average.

17. The system of claim 1, further comprising a motion sensor connected to the processing circuit.

18. The system of claim 17, wherein the processing circuit is further configured to:
detect motion exceeding a threshold, during a calibration measurement, and,
in response to the detecting of the motion exceeding the threshold, repeat the calibration measurement.

19. The system of claim 17, wherein the processing circuit is further configured to:
detect motion exceeding a threshold, during a speckle measurement, and,
in response to the detecting of the motion exceeding the threshold, initiate a calibration process.

20. The system of claim 1, wherein the array detector is a zero-degree chief ray angle detector.
